(19) United States
(12) Patent Application Publication
(10) Pub. No.: US 2024/0268421 A1
(43) Pub. Date: Aug. 15, 2024

(54) FOOD COLOURING AGENTS

(71) Applicant: v2food Pdy Ltd, Sydney (AU)

(72) Inventors: Jared RAYNES, Annandale (AU); Nicholas HAZELL, Sydney (AU); Peter RALPH, Forest Lodge (AU); Mathieu PERNICE, Waverley (AU)

(73) Assignee: v2food Pty Ltd, Sydney (AU)

(21) Appl. No.: 17/907,386

(22) PCT Filed: Mar. 31, 2021
(86) PCT No.: PCT/AU2021/050296

§ 371 Date: Sep. 26, 2022

(30) Foreign Application Priority Data
Mar. 31, 2020 (AU) 2020900989

(51) Int. Cl. A23L 5/46; A23J 3/14; A23J 3/22

(52) U.S. Cl. CPC A23L 5/46; A23J 3/14; A23J 3/227

(57) ABSTRACT

The present disclosure relates to food colouring agents. In particular, the disclosure relates to the use of linear tetrapyrrole-containing compounds, such as phycobiliproteins, for example, phycoerythrin, as colouring agents and/or metal ion carriers for the use in food products, such as in meat mimetic and meat replacement foods, and as ingredients therefor. The disclosure also relates to food products, such as meat mimetic and meat replacement food products, and ingredients therefor, comprising said colouring agents and/or metal carriers.

This application is a national phase application under 35 U.S.C. § 371 of International Application No. PCT/AU2021/050296, filed Mar. 31, 2021, the entire contents of which are hereby incorporated by reference. International Application No. PCT/AU2021/050296 claims benefit of Australian Application No. 2020900989, filed Mar. 31, 2020.

### FIELD

The present disclosure relates to food colouring agents. In particular, the disclosure relates to the use of linear tetrapyrrole-containing compounds, such as phycobiliproteins, for example, phycoerythrin, as colouring agents and/or metal ion carriers for the use in food products, such as in meat mimetic and meat replacement foods, and as ingredients therefor. The disclosure also relates to food products, such as meat mimetic and meat replacement food products, and ingredients therefor, comprising said colouring agents.

### BACKGROUND

With a need to feed a growing world-wide population, estimated to reach 9.7 billion people by 2050, there is a need to rebalance the animal derived component of the world's food sources to achieve sustainable food systems and increased food and nutrition security. Plant-derived alternatives to meat products represent a growing market due to the shifting dietary patterns of consumers. Increasingly, consumers are concerned about the impact the food production system has on the environment, climate change and animal ethics, and this influences the choices they make about food purchases. Vegans, vegetarians, and animal meat eaters who are reducing their consumption (reducitarians or flexitarians) are driving demand for meat alternatives made entirely or substantially from non-animal products.

Since the 1960s, textured soy protein products, prepared from soy flour or concentrate using extrusion technology have become a popular replacement for minced or ground meat. In more recent times, other non-animal protein sources, such as mycoproteins, mushrooms, legumes (e.g. peas, lupin, beans) and wheat, are also being manufactured into meat replacement or meat mimetic products

However, animal meat comprises a complex matrix of protein structures and fibres, within which are trapped fats, carbohydrates, water and other molecules, which contribute to the sensory, textural and structural characteristics (e.g. appearance, flavour, chewiness, juiciness) of the meat-containing food product, both in its raw and cooked states. While consumers may be willing to eliminate or reduce meat consumption for ecological or ethical reasons, many still prefer the meat replacement product to reproduce the “meat experience”: presenting and behaving like animal meat, not only in sensory aspects such as appearance, flavour and texture, but also in physical aspects like storage, handling and cooking. Methods of cooking and meal preparation is deeply culturally embedded and very resistant to rapid change. Given the complex structural and molecular make-up of animal meat, recreating the various characteristics so that a non-animal derived meat replacement product mimics or reproduces the raw and cooked characteristics of animal meat remains challenging.

One of the characteristics which is desirable to reproduce relates to the appearance of the meat replacement or mimetic product, in particular the colour, both in the raw (uncooked) and cooked states. Raw animal meat, such as chicken, beef, lamb or pork, is typically pink or red in colour, due to the presence of haemoglobin and myoglobin (iron and oxygen binding conjugated proteins responsible for transporting oxygen in the blood of vertebrates). However, upon cooking, for example, at a temperature of about 60-80° C., these proteins are denatured and the meat changes colour, losing its raw state pink or red colour, and typically turning white, brown and/or grey. This provides a visual indicator for the cook and aids to guide cooking time to achieve the desired flavour and/or texture of the cooked product, and in some circumstances an indicator of a food safety related heat treatment.

One option currently used to provide the pink or red colour to meat mimetic or replacement products is vegetable derived colouring agents, such as beetroot and radish extracts. However, while this affords a product that presents a pink or red colour for the meat mimetic or replacement product in the raw state, similar to animal meat, it does not follow through with the visual experience of the meat replacement or mimetic products turning white/brown/grey when cooked. Because there is no appreciable colour change upon cooking, the meat can be overcooked by the cook in order to achieve the desired cooked colour. There remains a need for colouring agents for meat replacement and mimetic products that behave in a visual manner similar to the colour change observed when animal meat is cooked.

Phycobiliproteins (PBPs) are highly water soluble fluorescent proteins found in cyanobacteria (blue green algae), certain eukaryotic micro and macro algae, such as red algae (Rhodaphyta), some cryptophytes and dinoflagellates, comprising protein chains covalently bound to linear tetrapyrrole chromophores (known as bilins). Assembled into membrane-extrinsic molecular superstructures, referred to as phycobilisomes, they serve as light-harvesting pigments by acting as antennae for the capture and transfer of light energy, which is otherwise inaccessible to chlorophyll.

The phycobiliproteins making up the phycobilisomes are arranged in two structurally distinct units: core and rods, comprising cylinders of stacked discs of trimers (core) or hexamers (rods) of αβ subunits. The αβ subunit is itself a heterodimer are made up of α and β polypeptide chains (approximately 160-180 amino acid residues each) covalently linked to linear (non-cyclic) tetrapyrrolic chromophores, which confer the light absorption properties.

Based on their absorption properties, phycobiliproteins are commonly classified into four subclasses: blue-coloured phycocyanins (typical λₘₐₓ=610-620 nm), deep red/pink-coloured phycoerythrins (typical λₘₐₓ=540-570 nm), bluish green-coloured allophycocyanins (typical λₘₐₓ=650-655 nm) and the less frequently found magenta-coloured phycoerythrocyanins (typical λₘₐₓ=560-600 nm). Phycobiliproteins may be further categorised by prefixes, depending on their origin: e.g., C for Cyanobacteria, R for Rhodophyta, and B for Bangiales, although specific phycobiliprotein types are not always restricted to specific taxa.

Examples of some typical absorbance and emission values are presented below in Table 1.

TABLE 1
Exemplary Absorbance and Emission Values of Phycobiliproteins.

| | | | λₘₐₓ | λ_{shoulder} | Emₘₐₓ |
|---|---|---|---|---|---|
| Protein | Colour | Prefix | (nm) | (nm) | (nm) |
| Phycocyanin (PC) | Blue | C | 620 | — | 640 |
| | | R | 555/617 | — | 636 |
| Phycoerythrin (PE) | Red/Pink | R | 565/540 | 498 | 575 |
| | | B | 565/545 | 499 | 576 |
| | | C | 560 | — | 577 |
| | | b | 545 | 565 | 570 |
| Allophycocyanin | Bluish/green | | 618/671 | — | 675 |
| (APC) | | | | | |
| Phycoerythrocyanin | Magenta | | 568/570 | 595 | 625 |
| (PEC) | | | | | |

The four types of chromophore that confer these properties are phycoerythrobilin (PEB), phycouribilin (PUB), phycocyanobilin (PCB) and phycobiliviolin (PXB) (see Scheme 1 below—pictured in the context of linkage to a peptide via disulphide bonds). Differences in the π-electron conjugation are responsible for their absorption properties and colour.

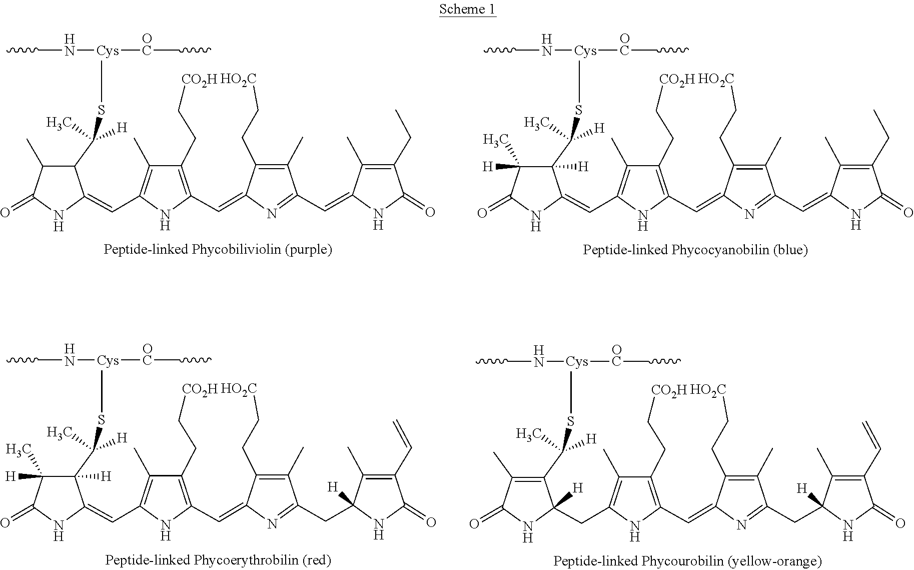

The scientific literature is replete with characterizations of phycobiliproteins, and some common themes may be found amongst the subclasses. Phycoerythrocyanin exists in trimeric (αβ)₃ or hexameric (αβ)₆ form, with a PXB chromphore attached to the α polymer chain and two PCB chromophores attached to the β chain. Allophycocyanin is in the form of a trimer, with both the α and β subunits possessing one PCB chromophore each. Phycocyanin can exist in trimeric or hexameric form with the α subunit possessing one PCB chromophore and, depending on the species, two PCB chromophores or one PCB and one PEB chromophore on the β subunit. b-Phycoerythrin and C-phycoerythrin occur in oligomeric forms of the αβ subunit (n, 3 or 6) with the α subunit possessing two PEB chromophores and the β subunit bearing 3 (b-) or 4 (C-) PEB chromophores. R-Phycoerythrin and B-phycoerythrin, the most abundant forms found in red algae (Rhodophyta), commonly comprise the hexameric αβ subunit and an additional linking γ subunit: (αβ)₆γ. The α subunit of both the R- and B-forms possess two PEB chromophores, whereas the β subunits bears two PEB chromophores and one PUB chromophore (R-phycoerythrin) or three PEB chromophores (B-phycoerythrin). The γ subunit of R-phycoerythrin bears two PEB chromophores and two PUB chromophores, whereas the γ subunit of B-phycoerythrin bears four PUB chromophores. (Dumay, J. et al, Phycoerythrins: Valuable Proteinic Pigments in Red Seaweeds, Chapter 11, Advances in Botanical Research, Vol 71, pp 321-343, 2014, Elsevier Ltd, and references cited therein; and Jiang, T., et al, PROTEINS: Structure, Function, and Genetics 34:224-231 (1999) and references cited therein).

Notwithstanding the above, spectroscopic differences even between the same type of phycobiliproteins, such as phycoerythrins, are observed. For example, spectroscopic differences between phycoerythrins reflect the content and ratio of PEB and PUB chromophores (see, for example, Klotz A. V., and Glazer, A. N, The Journal of Biological Chemistry, 260, 4856-4863, 1985), and elsewhere it has been shown that phycoerythrins purified from various Rhodophyta species may exhibit differing UV visible absorption spectroscopic characteristics. (See for example, Rennis, D. S., and Ford, T. W., A survey of antigenic differences between phycoerythrins of various red algal (Rhodophyta) species, Phycologica, (1992), 31, 192-204); and Ma, J, et al, Nature, 2020, 579, 146-151). In particular, while some phycoerythrins exhibit absorbance peaks at approximately 495-503 nm and 540-570 nm, is has been shown that phycoerythrin extracted from some species demonstrate reduced or absent peaks at approximately 495-503 nm in the UV visible spectrum (see Rennis and Ford, supra, page 197, FIG. 1; and Ma et al, supra, Extended Data FIG. 1). This is attributed to a lower PUB content (Ma, J, et al, supra,). Each of the α, β and γ subunits of a phycobiliprotein, such as phycoerythrin, may also have differing absorbance profiles (see Tamara et al, Chem 5, 1302-1317, 2019), and may thereby contribute to spectroscopic differences between phycroerythrins. Elsewhere it is reported that C-Phycoerythrin (Schizothrix calicola) demonstrates a major absorption maxima in the visible regions at 565 nm (PEB); R-Phycoerythrin (Ceramium rubrum) demonstrates a major absorption maxima in the visible regions at 567 nm (PEB)>538 nm (PEB)>498 (PUB); and B-Phycoerythrin (Porphyridium cruentum) demonstrates a major absorption maxima in the visible regions at 545 nm (PEB)>563 nm (PEB)>498(S) (PUB) (Glazer, A. N., and Hixson, C. S., The Journal of Biological Chemistry, 250. 5487-5495, 1975).

Thus, the exact number and nature of the protein subunits and chromophores of a phycobiliprotein (and thus the absorbance spectroscopic characteristics of the phycobiliprotein) and the amount produced, are species-dependent, and can be further influenced by the growth conditions (e.g. light, temperature, nutrients, pH etc), and may therefore result in physical and spectroscopic property differences, even within a single phycobiliprotein subclass.

Phycobiliproteins exhibit intense fluorescent properties and find many applications in biotechnology, such as in fluorescence-based techniques and immunoassays. They are also used as food colouring agents in the food industry, with phycocyanin from Spirulina (Arthrospira platensis) used as a blue colouring agent (e.g. in gums, sorbets, ice cream, candies, soft drinks and dairy products), and B- and b-phycoerythrins extracted from P. cruentum reported as used as red colouring agents in jelly desserts and dairy products (Dumay, J. et al supra).

### SUMMARY

It has now surprisingly been found that certain phycobiliproteins, (e.g. phycoerythrin), when present in a food product that is to be cooked, such as a meat mimetic or meat replacement food product can visually afford a similar colour change to that which occurs during cooking of animal meat, e.g. a change in colour from a red or pink (“raw” state) to white, brown and/or grey (“cooked”). The use of phycobiliproteins, such as phycoerythrin, as a colouring agent in meat mimetic and meat replacement products, can thus provide the consumer with a visual colour cooking experience that mimics the cooking of animal meat.

In a first aspect, there is provided a meat mimetic food product comprising one or more phycobiliproteins, in an amount sufficient to visually confer a pink or red colour to the food product, and which affords a visual colour change upon cooking of the food product to an internal temperature in the range of about 50-95° C., such as about 60-85° C.

Another aspect provides use of one or more phycobiliproteins in the manufacture of a meat mimetic food product, wherein the one or more phycobiliproteins are included in the food product in an amount sufficient to confer a visual pink or red colour to the food product, and subsequently afford a visual colour change upon cooking of the food product to an internal temperature in the range of about 50-95° C., such as about 60-85° C.

Another aspect provides a cooked meat mimetic food product of the disclosure.

In some embodiments of the above, the one or more phycobiliproteins comprise at least phycoerythrin, such as R-phycoerythrin and/or B-phycoerythrin and/or C-phycoerythrin and/or b-phycoerythrin. In further embodiments, the one or more phycobiliproteins further comprise one or more of phycocyanin, allophycocyanin, and phycoerythrocyanin. In still further embodiments, phycoerythrin comprises at least 50% by weight of the one or more phycobiliproteins, such as at least 80%, 90% or 95%. In further embodiments, the one or more phycobiliproteins consists essentially of phycoerythrin.

In some embodiments, the temperature at which a loss of 50% absorbance of λₘₐₓ is observed for the phycobiliprotein is in the range of about 50-95° C., more preferably in the range of about 60-85° C. In further embodiments, the λₘₐₓ is in the range of about 540-570 nm, such as about 545-565 nm, or 550-560 nm. In other embodiments, the λₘₐₓ is in the range of about 495-503 nm

In some embodiments, phycoerythrin, may be obtained from one or more suitable algal species and is included in the food product in an extracted, purified (e.g at least 90%, 95% or 99% purity), or at least partially purified form (e.g. greater than 50% purity, such as greater than 60%, 70% or 80% purity. In some embodiments, the one or more phycobiliproteins is included in the food product as an algal form and may be included as whole or macerated algae, which can be wet (e.g. as a paste, suspension or slurry in water, in liquid or frozen state) or dry (e.g. dried by heat, evaporation or freeze drying).

In any one or more aspects or embodiments described above, the meat mimetic food product comprises a non-animal protein source, one or more carbohydrates, one or more fats and oils (preferably vegetable derived, i.e. non-animal), one or more flavour components and water. Other components, such as thickeners, binders and preservatives, may be added. In further embodiments thereof, the meat mimetic product comprises soy protein, such as textured soy protein or other plant-based proteins, such as faba bean, pea, wheat, chickpea and mungbean.

In any one or more aspects or embodiments described above, the meat mimetic food product may be a poultry (e.g. chicken), beef, veal, lamb, pork, goat, kangaroo, or fish/seafood meat mimetic food product. In some further embodiments, the meat product is a ground meat mimetic food product.

It has been found that certain phycobiliproteins, such as phycoerythrin, have the ability to chelate with a metal ion, such as iron (Fe), and to increase ferritin production, and thus may also provide a convenient mechanism for metal ion delivery in a food product, particularly iron delivery as a nutritional benefit. Thus, in one or more aspects or embodiments, described above, the one or more phycobiliproteins may be chelated or co-ordinated to a metal ion, such as iron Fe²⁺ or Fe³⁺. In some embodiments, the metal ion may be preliminarily co-ordinated with the at least one phycobiliprotein by premixing the metal ion (e.g., as a solution) with the phycobiliprotein before adding to the meat mimetic food product mixture. In other embodiments, the metal ion (e.g., as a solution) and the one or more phycobiliproteins may be added to the meat mimetic food product mixture as separate components, simultaneously or sequentially.

### BRIEF DESCRIPTION OF THE FIGURES

FIG. 1 depicts the DSF fluorescence signal obtained by heating 100 μL clarified phycoerythrin samples from 25-95° C.

FIG. 2 depicts the UV/VIS absorbance spectra of phycoerythrin extracts before and after heating at 95° C. for 6 min

FIG. 3 depicts the UV/VIS absorbance spectrum of clarified phycoerythrin extracts obtained from Porphyridium purpureum, Asparagopsis taxiformis, Bonnemaisonia hamifera and wild red seaweed.

FIG. 4 depicts the thermal denaturation of phycoerythrin extracts obtained from Porphyridium purpureum, Asparagopsis taxiformis, Bonnemaisonia hamifera and wild red seaweed from 20-95° C. at 536 nm.

FIG. 5 depicts a UV/VIS absorbance spectrum at room temperature of phycoerythrin extract (before and after heating from 20° C. to 95° C.) prepared from Rhodomonas salina red microalgae biomass grown in culture.

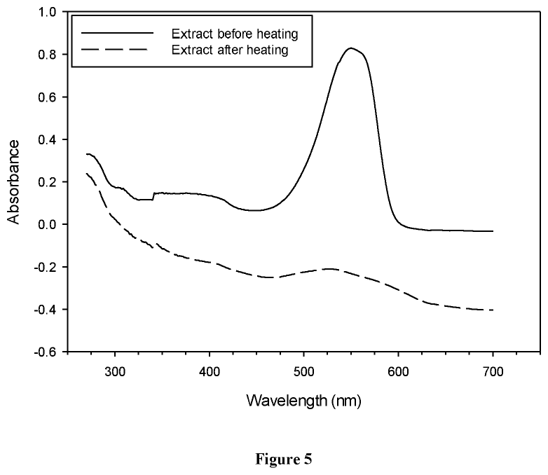

FIG. 6 depicts a temperature scan from 20° C. to 95° C. at 550 nm, of phycoerythrin extract obtained from Rhodomonas salina red-microalgae grown in culture.

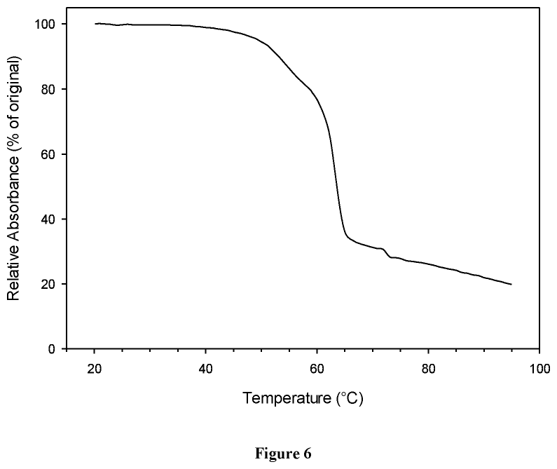

FIG. 7 depicts fluorescence emission spectra for phycoerythrin extract with increasing concentrations of iron (II) chloride.

### DESCRIPTION

Throughout this specification and the claims which follow, unless the context requires otherwise, the word “comprise” and variations such as “comprises” and “comprising” will be understood to imply the inclusion of a stated integer or step or group of integers but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the invention defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The singular forms “a”, “an” and “the” include plural aspects unless the context clearly dictates otherwise.

The term “invention” includes all aspects, embodiments and examples as described herein.

As used herein, “about” refers to a quantity, value or parameter that may vary by as much as 25%, 20%, 15%, 10%, 5%, or 1-2% of the stated quantity, value or parameter, and includes at least tolerances accepted within the art. When prefacing a recited range or list of values, it is intended to apply to both upper and lower limits of the range and each member of the list.

Unless the context indicates otherwise, features described below may apply independently to any aspect or embodiment of the invention

As used herein, a meat mimetic food product (also known as meat analogue, meat alternative or meat replacement) refers to a non-animal protein-containing food product that mimics, resembles or performs in a manner similar to an animal-derived meat product in any one or more physical or sensory factors, including pertaining to appearance, taste, texture, mouthfeel (moistness, chewiness, fattiness etc), aroma, or other physical properties, including structure, texture, storage, handling, and/or cooking. In some embodiments the protein may be plant or fungal derived. In some embodiments, the meat mimetic product is a plant-based food product.

In some embodiments, the meat-mimetic food product comprises a non-animal protein source, and does not contain or include, or does not substantially contain or include (i.e. less than about 5% w/w, such as less than about 4% w/w, or less than about 3% w/w or less than about 2% w/w or less than about 1% w/w), any ingredient derived or obtained from animal sources. However, it is to be understood that the present disclosure is not so limited, and in other embodiments, meat mimetic food products or ingredients therefor may contain a proportion of one or more animal derived ingredients, including any one or more of egg, casein, whey, muscle, fat, cartilage and connective tissue, offal or blood, or components thereof, for example in an amount of about 5%, 10%, 15% 20%, 25%, 30%, 35%, 40%, 45% or 50% by weight of the food ingredient or meat mimetic food product. This may include those cultured meat mimetic products that contain cell-based meat grown from stem cell cultures.

As used herein, a “pink” or “red” colour, when used in relation to a raw or uncooked meat mimetic food product, refers to a pink or red colour that is visually similar to the corresponding animal meat form, including: pink colours, such as corresponding to chicken pork, veal or goat; pink/orange or red/orange colours, such as corresponding to salmon; and red colours, such as corresponding to lamb, mutton, beef or kangaroo meat.

As used herein, “colour change”, when used with reference to cooking of a meat mimetic product, refers to the visual reduction in pink/redness of the product, and the corresponding appearance of white, brown, or grey colour, reflecting the denaturation of the one or more phycobiliproteins.

The terms “cooking” and “cooked” refer to the application of heat, for example by frying, baking, roasting, grilling, broiling, sautéing, barbecuing, steaming, simmering, boiling, microwaving, etc. In some advantageous embodiments, the at least one phycobiliprotein thermally denatures such that there is an observed colour shift from a pink or red colour to white, brown, or grey at a temperature that corresponds approximately to the temperature or temperature range at which a similar colour shift occurs in animal meat. In some embodiments, the food product (e.g. meat mimetic or replacement) is cooked to at least an internal temperature in the range of about 50-95° C., such as about 55-90° C. or about 60-85° C. In some further embodiments, the food product is cooked to an internal temperature in the range of about 60-65° C., or about 65-70° C., or about 70-75° C. or about 75-80° C. In further embodiments, the food product may be cooked to an internal temperature of about 65, 66, 67, 68, 69, 70, 71, 72, 73, 74, 75, 76, 77, 78, 79, 80, 81, 82, 83, 84 or 85° C.

The one or more phycobiliproteins for use in the present disclosure singularly, or together, afford a pink or red colouration to the raw or uncooked meat mimetic food product. Advantageously, at least one or more phycobiliproteins are visually pink or red in colour. Thus, in some embodiments of the above, the one or more phycobiliproteins comprise at least phycoerythrin, which typically demonstrates λₘₐₓ in the range of about 540-570 nm, such as about 540, 545, 550, 555, 560, 565 or 570 nm (attributed to the PEB chromophore), and optionally a peak or shoulder in the range of about 495-503 nm, such as about 495, 496, 497, 498, 499, 500, 501, 502 or 503 nm (attributed to the PUB chromophore) (Klotz A. V., and Glazer, A. N., supra). Examples of phycoerythrin include R-phycoerythrin and/or B-phycoerythrin and/or C-phycoerythrin and/or b-phycoerythrin.

In further embodiments, the one or more phycobiliproteins include phycoerythrin and may further comprise one or more of phycocyanin, allophycocyanin, and phycoerythrocyanin. Thus, in some embodiments the one or more phycobiliproteins may include phycoerythrin and at least phycocyanin. In some embodiments the one or more phycobiliproteins may include phycoerythrin and at least allophycocyanin. In some embodiments the one or more phycobiliproteins may include phycoerythrin and at least phycoerythrocyanin. In some further examples the one or more phycobiliproteins may include phycoerythrin and at least two other phycobiliproteins. In some embodiments thereof, phycoerythrin is present in a dominant amount (on a w/w basis) compared to any other phycobiliprotein, or all other phycobiliproteins, for example the one or more phycobiliproteins comprises at least 50%, or at least 55%, or at least 60%, or at least 65% or at least 70%, or at least 75%, or at least 80%, or at least 85%, or at least 90% or at least 95%, or at least 96%, or at least 97%, or at least 98%, or at least 99% or 100% of phycoerythrin.

Micro (unicellular) and macro (multicellular) algae can provide a convenient source of phycobiliproteins, Suitable sources, may be, for example: cyanobacteria (Cyanophyceae), such as Arthrospira (Spirulina) sp, and Anabaena sp; red algae (Rhodophytes—Rhodophyceae), such as Gracilaria sp and Porphyridium sp; and cryptophytes (Cryptophyceae), such as Rhodomonas sp (e.g Rhodomonas salina). Sources of phycobiliproteins, such as phycoerythrin, include naturally occurring or genetically modified species. Phycobiliproteins, such as phycoerythrin, may also be obtained through recombinant techniques and methods known in the art. Algae species that do not naturally produce high amounts of phycoerythrin may still provide a suitable source of phycoerythrin. For example, species such as Arthrospira sp may, through mutagenesis and directed evolution, together with appropriate growing conditions, produce increased quantities of phycoerythrin.

The at least one or more phycobiliproteins may be derived from a single source, or a combination of sources. As one example, red-coloured phycoerythrin may be obtained from one or more red algae and/or cyanobacteria and/or cyrptophyte sources, and may optionally be combined with one or more other, same or different, phycobiliproteins obtained from a different source(s).

The amount and type(s) of phycobiliproteins produced by phycobiliprotein-producing organisms, for example, cryptophyte, cyanobacteria and/or red algae, may be manipulated by culture conditions, e.g., nutrients, carbon source, pH, temperature and exposure to differing light conditions, to, for example, increase the total amount of phycobiliproteins produced, and/or skew the production of one or more phycobiliproteins/chromophores over another. For example, phycoerythrin production can be increased by culture of algae under green light, whereas in some embodiments, with culture under red light more phycocyanin is produced. Methods therefor are known in the art, for example as described in some of the references supra, and in Hsieh-Lo, M., et al, Algal Research, 42 (2019) 101600; Ferreira, R., et al, Food Sci. Technol, Campinas 35(2): 247-252, Abr.-Jun.2015; Oostlander, P. C., et al, Algal Research, 47 (2020), 10189; and Minh Thi Thuy Vu, et al, Journal of Applied Phycology, 28, 1485-1500 (2016), and the references cited therein, the contents of which are incorporated herein by reference.

In some embodiments, the algal biomass is rich in, or presents a high proportion of the desired phycobiliprotein, such as. phycoerythrin. Thus, in some embodiments, the algal source biomass contains from about 5 mg to about 150 mg phycoerythrin per 1 g dry weight, for example, about 5-50 mg phycoerythrin per 1 g dry weight. In further examples the algal source biomass contains about 10 mg, or about 15 mg or about 20 mg, or about 25 mg or about 30 mg, or about 35 mg or about 40 mg, or about 45 mg or about 50 mg, or about 55 mg or about 60 mg, or about 65 mg or about 70 mg, or about 75 mg or about 80 mg, or about 85 mg or about 90 mg, or about 95 mg or about 100 mg, or about 105 mg or about 110 mg, or about 115 mg or about 120 mg, or about 125 mg or about 130 mg, or about 135 mg, or about 140 mg, or about 145 mg phycoerythrin per 1 g dry weight.

Phycoerythrin content of algae can be determined using methods known in the art, (see for example, Gnatt E., and Lipschultz C. A., Biochemistry, 1974, 13, 2960-2966; Kursar T. A., & Alberte R. S., Plant Physiology, 1983, 72, 409-414; Sobiechowska-Sasim, M., et al, J Appl Phycol (2014) 26:2065-2074; and Saluri M., et al, Journal of Applied Phycology, 32, 1421-1428, 2020). In some embodiments, R-Phycoerythrin content can also be quantified using the method of J. Dumay et al, “Extraction and Purification of R-phycoerythrin from Marine Red Algae” by Justine Dumay, Michèle Morançais, Huu Phuo Trang Nguyen, and Joel Fleurence in “Natural Products From Marine Algae: Methods and Protocols, Methods in Molecular Biology, vol. 1308”, by Dagmar B. Stengel and Solène Connan (eds.), Springer Science Business Media New York 2015.

The one or more phycobiliproteins are added to, or are present in, a meat mimetic food product in any amount and combination that provides the desired colour, preferably a red or pink colour that mimics the colour of the corresponding raw animal meat, for example, beef, veal, lamb, pork, goat kangaroo, fish (e.g. salmon, trout, tuna) and poultry (e.g. chicken, duck, goose, turkey, and game birds).

In one or more embodiments, the one or more phycobiliproteins are incorporated into the meat mimetic food product as an extract or at least partially purified form obtained from an algal source.

Some exemplary methods for obtaining phycobiliprotein-containing extracts such as phycoerythrin-containing extracts, are described in RU 2548111C1, CN101139587A, JP2017532060A, CN1796405A, CN101617784A, CN1618803A, CN101942014A, WO2003099039A1, as well as references cited herein, the contents of which are incorporated herein by reference.

The exact colour of a phycobiliprotein (e.g. phycoerythrin), is dependent on the species from which it is obtained, the number and nature of protein subunits, and the number and nature of chromophores present. The presence or absence of additional compounds, such as other phycobiliproteins (e.g. allophycocyanin and/or phycocyanin) may also affect the overall visual colour.

One exemplary general method for the preparation of the colouring agents of the disclosure includes the steps of homogenizing a phycobiliprotein-containing biomass, such as a red algae or blue-green algae (cyanobacteria), or cryptohyte, in aqueous solution (e.g. water or a buffer solution). Optionally, the liquid containing extracted one or more phycobiliproteins may be separated from the solid material. Optionally, the homogenized biomass or separated phycobiliprotein-containing aqueous suspension or solution may be concentrated and/or dried. The process may include further optional steps such as ultrasound treatment (sonication) of the homogenized biomass to enhance extraction of phycobiliproteins into the aqueous phase.

In some preferred embodiments, the phycobiliproteins are extracted phycobiliproteins, obtained by at least one extraction or separation step from a phycobiliprotein containing biomass such as cryptophytes, cyanobacteria (blue-green algae), or macro or micro red algae (Rhodophyta).

Thus, in some embodiments, the at least one phycobiliprotein may be added to the food product, (e.g. meat mimetic product) in the form of a macerated biomass, for example in which an appropriate biomass, such as cyanobacteria and/or red algae, is homogenized in water or an aqueous solution (e.g. buffer solution (for example sodium or potassium phosphate or sodium or potassium acetate solution), at a pH in the range of about 6.5 to about 7.5, e.g. a pH of about 6.6, 6.7 6.8, 6.9, 7.0, 7.1, 7.2, 7.3 or 7.4), such that phycobiliproteins are extracted into the water or aqueous solution. In some embodiments the resulting suspension or slurry (optionally having been further treated with ultrasound) can be added directly to the ingredients of the food product, such as a minced or ground meat mimetic/replacement mixture. In further embodiments, the homogenized slurry or suspension (optionally having been further treated with ultrasound) may be further concentrated or dried before addition to the food product. In some of these embodiments, the addition of biomass solid material to the food product may advantageously increase the nutritional value of the food product and/or introduce a flavour component (e.g. umami flavour due to the presence of glutamate), or a flavour precursor (e.g. glutathione or other amino acid) that helps create cooked meat flavour during subsequent cooking of the meat mimetic, to the final flavour profile.

In some embodiments, the homogenized material (optionally further treated by ultrasound) can be further purified to a desired level of purity by separation and removal of some or all solid material, using any one or more suitable separation techniques such as sieving, centrifugation, precipitation, filtration, ultrafiltration, microfiltration, nanofiltration, diafiltration, reverse osmosis, and chromatography to afford an aqueous suspension or solution of one or more phycobiliproteins. The resulting solution may be optionally further concentrated to a desired concentration before adding to the food product.

In some embodiments, an extract solution comprising one or more phycobiliproteins may be further subjected to an appropriate separation step, such as dialysis or reverse osmosis treatment, in order to remove any metals and/or other impurities present.

In one or more embodiments, the phycobiliprotein extract suspension or solution may be subjected to one or more freezing steps, e.g. at about −10° C. or below, such as about −15° C. or below, or about −20° C. or below, or −25° C. or below.

In some further embodiments, an aqueous solution comprising one or more phycobiliproteins may be dried to form a solid material by any suitable drying technique, such as evaporation, freeze drying, spray drying or supercritical drying. In some embodiments, the at least one phycobiliprotein may be added to a food product, such as a meat mimetic or replacement, in dried form or equal to a dry weight amount of from about 0.5 mg/g to about 25 mg/g, such as about 1, 2, 3, 4, 5, 6, 7, 8, 9, 10, 11, 12, 13, 14, 15, 16, 17, 18, 19, 20, 21, 22, 23, or 24 mg/g.

Liquid phycoerythrin extracts may optionally be pasteurised by heating the liquid to a temperature below that at which the proteins denature and change colour, e.g. less than about 80° C., or less than about 77° C., or less than about 75° C. or less than about 70° C.

In some embodiments, one or more phycobiliproteins is included in the food product as an algal form (e.g. a species of Rhodaphyta, cyanobacteria or Cryptophyte) and may be added as a whole algal biomass, optionally macerated (for example by one or more freeze thaw cycles and/or in a homogenizer). In some embodiments, the algae is microalgae. The algae may drained and/or filtered and used wet (e.g. as a paste, suspension or slurry in water/culture medium), for example about 0.1% w/w biomass, or about 0.5% w/w biomass, or about 1% w/w biomass, about 5% w/w biomass, or about 10%, w/w biomass or about 20%, w/w biomass, or about 30% w/w biomass, or about 40% w/w biomass or about 50%, w/w biomass, or about 60%, w/w biomass, or about 70%, w/w biomass, or about 80% w/w biomass or about 85% w/w biomass, or about 90% w/w biomass, or about 95% w/w biomass, or greater). The algal biomass may be used directly or, optionally may be chilled, frozen and/or pasteurized before further use. In other embodiments, the algal biomass may be dry (e.g. dried by heat, evaporation or freeze drying).

The algal biomass may be added to the meat mimetic product in an amount suitable to impart the desired pink or red colour. The amount of algal biomass to be added may be dependent on the phycoerythrin content of the algae. In some embodiments, the algal biomass is added in an amount no more than about 20% dry weight per weight of meat mimetic product. In some embodiments the algal mass may be added to the meat mimetic product in the range of about 0.1% to about 20% dry weight per weight of food product, such as from about 0.1-5%, for example about 0.1%, 0.25%, 0.5%, 0.75%, 1%, 1.5%, 2%, 2.5%, 3%, 3.5%, 4%, 4.5%, 5%, 6%, 7%, 8%, 9%, 10%, 11%, 12%%, 13%, or 14% or 15%, or 16%, or 17% or 18% or 19% on a dry weight per weight of the food product basis.

Advantageously, in some embodiments, algal biomass has a phycoerythrin content such that it can be used in amounts that are sufficient to provide the desired red or pink colouring to a meat mimetic food product, but does not impart adverse marine flavour to the meat mimetic food product (mostly due to the presence of dimethyl sulphide). This can be determined by sensory evaluation, which can be performed by a taste tester comparing the taste of meat-mimetic samples with and without phycobiliprotein ingredients. Suitable phycoerythrin contents therefor may be in the range of 5 mg to about 150 mg phycoerythrin per 1 g dry weight, such as about 10-50 mg, as described supra. In some further embodiments, the algal biomass is a (unicellular) microalgae. Suitable examples may include: Porphoridium sp (e.g. P. purpureum and P. sordidum), Rhodochaete sp. (e.g. R. parvula), Hildenbrandia sp. (e.g. H. rivularis), Erythrotrichia sp. (e.g. E. carnea), Rhodella sp. (e.g. R. violacea), Rhodosorus sp. (e.g. R. marinus), Arthrospira sp (e.g. A. platensis), Fremyella sp. (e.g. F. diplosiphon) or Rhodomonas sp, (e.g. R. salina). One preferred algal source is R. salina. In a further embodiment, the algae is R. salina strain CS-174.

Algal species and strains may be obtained from commercial sources and Culture Collections (e.g. CSIRO Australian National Algae Culture Collection, UTEX Culture Collection, CCAC, Germany; NIVA, Norway). Methods for culturing algae, such as those species described above, are known in the art. Some methods are described in Oostlander, P. P., et al, Algal Research, 47, 101889, 2020; Minh Thi Thuy Vu, et al, Journal of Applied Phycology, 28, 1485-1500 (2016); Guevara, M., J. Appl. Phycol., 28(5), 2651-2660, 2016 and references cited therein, the contents of which are incorporated herein by reference.

In some embodiments, the suitability of the at least one phycobiliproteins (e.g. phycoerythrin) for use in the present disclosure can be determined by evaluating the UV/VIS absorbance spectrum of an extracted or purified phycobiliprotein, and/or determining the temperature at which the phycoerythrin denatures, such as determining the temperature at which 50% loss of absorbance of λₘₐₓ is observed, a desirable temperature being in the range of 50-95° C.

For example, a phycobiliprotein extract can be obtained according to any one of the processes described herein, or other method known in the art, and its UV/VIS absorbance spectrum obtained and evaluated for the presence of characteristic peaks e.g. λₘₐₓ at 540-570 nm, and optionally a further peak or shoulder at 495-503 nm, for phycoerythrin.

Thus, the suitability of an algal species to provide a desirable red or pink colour, for use as either a source of at least partially isolated or purified phycoerythrin, or for use in whole or macerated form, may be determined by UV/VIS absorbance spectrum for an extracted, isolated or at least partially purified sample of phycoerythyrin obtained from the algal species.

The extracted or isolated or at least partially purified form of phycoerythrin may advantageously exhibit a 540-570 nm to 495-503 nm UV/visible absorbance peak ratio of at least 1:1, such as at least 1.5:1, or at least 2.0:1, or at least 2.5:1 or at least 3:1, or at least 4:1, or at least 5:1, or at least 6:1, or at least 7:1, or at least 8:1, or at least 9:1, or at least 10:1. In some embodiments, the UV/VIS absorbance spectrum essentially demonstrates only a maximum peak/shoulder at about 540-570 nm (corresponding to PEB), such as at about 550-565 nm, and at about 280-290 nm (corresponding to protein), thus reflecting high levels of PEB in the phycoerythrin sample.

The suitability of at least one phycobiliproteins (e.g. phycoerythrin) for use in the present disclosure (added to the meat mimetic product either as an extract or at least partially purified form, or in the form of whole or macerated algae) may be determined by assessing the degree of reduction of λₘₐₓ upon heating. Thus, the temperature at which a loss of at least about 50% absorbance at the λₘₐₓ wavelength is observed, may be indicative of the approximate temperature at which a corresponding visual colour change may be observed when cooking the meat mimetic food product. In some preferred embodiments, the temperature at which a loss of 50% absorbance of λₘₐₓ (e.g. at 540-570 nm) is observed is in the range of about 50-95° C., more preferably in the range of about 60-85° C. In further embodiments, the λₘₐₓ is in the range of about 545-565 nm, such as about 550-560 nm.

Phycobiliproteins have been shown to chelate or co-ordinate with metal ions, such as Fe², (see Example 4 herein, and Sonani, R. R., et al, Process Biochemistry 49 (2014) 1757-1766). It has now been demonstrated that the presence of a phycobiliprotein, such as phycoerythrin, can improve the bioavailability of a metal ion, such as iron, by promoting production of ferritin, a protein that stores iron in the body and releases it throughout the body in a controlled fashion, thereby acting as a buffer against iron deficiency and overload. When used in a meat mimetic food product, this can afford a food product that may provide the body with a valuable source of iron.

Accordingly, in some embodiments, one or more phycobiliproteins used in accordance with the present disclosure may also act as carrier proteins for metal ion, such as Fe²⁺ or Fe³⁺ delivery. In one or more embodiments, there is provided a metal-chelated (e.g. Fe²⁺ or Fe³⁺) phycobiliprotein for use in the preparation of a meat mimetic food product, as well as a raw or cooked meat mimetic food product comprising said metal-chelated phycobiliprotein.

In some embodiments, the iron is provided in its 2+ oxidation state (for example as ferrous chloride (FeCl₂) or iron sulfate (e.g. FeSO₄, and hydrates thereof, such as FeSO₄·7H₂O)). In some embodiments, the iron is provided in its 3+ oxidation state, such as ferric chloride (FeCl₃).

The iron compound may be used with the one or more phycobiliproteins in a molar ratio of Fe:phycobiliprotein(s) of about 1:10 to about 3:1, for example, about 1:5, 1:2, 1:1.5, 1:1, 1.5:1 or 2:1. In further embodiments, the molar ratio of Fe:PE is about 1:10 to about 3:1, for example, about 1:5, 1:2, 1:1.5, 1:1, 1.5:1 or 2:1.

In some embodiments, the at least one phycobiliprotein colouring agent may also be used in conjunction with one or more additional colouring agents, either separately added to the food product, or combined with the one or more phycobiliproteins to form a mixture of colouring agents, and then added to the food product. In some embodiments, the one or more additional colouring agents excludes agents that contain a cyclic tetrapyrrole (and pyrrole-like) moiety, such as porphyrins, chlorins, bacteriochlorins, corroles and corrins, and metal complexes thereof, e.g. protoporphyrin IX and haem, and their protein conjugates. In some embodiments, the meat mimetic food product, in raw and/or cooked forms, excludes such separately added cyclic tetrapyrrole-containing compounds. It will be understood that phycobiliproteins added in algal form will contain native or endogenous cyclic chlorophyll, and the above certain embodiments are not to be construed as excluding the presence of a cyclic tetrapyrrole and pyrrole-like moieties which are endogenous to and inherently present in the algal species from which the phycoerythrin is derived.

In some embodiments, the colouring agent for the meat mimetic product consists of or consists essentially of one or more phycobiliproteins. In some embodiments, the colouring agent consists of or consists essentially of phycoerythrin.

In some embodiments, the one or more additional colouring agents are non-animal and non-coal/tar derived, and thus are suitable for vegetarian or vegan consumers. Appropriate colours may include one or more of red, magenta, purple/violet, orange, yellow, brown, blue and green. Some exemplary plant-derived colouring agents may include anthocyanins, betalains, carotenoids, flavonoids, and polyphenols. In some embodiments such colouring agents may be added as a juice, concentrate, extract or dried powder form derived from plants, such as berries, grapes, beetroot, radish, turmeric and carrot. Other additional colouring agents may include brown colours such as caramel/burnt sugar.

Meat mimetic food product may include one or more of non-animal protein sources such as soy protein, (e.g. texturized soy protein, soy protein isolate), pea protein, faba bean protein, lupin protein, mung bean protein, legumes (such as peas, beans (e.g. black beans, kidney beans, cannellini beans, pinto beans, mung beans), lupin, chick peas lentils), nuts, seeds, mushrooms and other fungal sources (e.g. Fusarium venenatum), and algae and microbial sources; one or more carbohydrate sources, such as sugars, including, monosaccharides and disaccharides (e.g. glucose, fructose, arabinose, ribose, maltose sucrose, dextrose maltodextrin, xylose, lactose, arabinose), oligosaccharides, polysaccharides, starches, gums, carrageenans, pectins, and fibres; one or more of fats and oils (e.g. plant derived oils, such as canola, sunflower, olive, coconut, vegetable, palm, peanut, flaxseed, cottonseed, corn, safflower, rice bran oil), emulsifiers (e.g. lecithin, polysorbates (20, 40, 60 80)); binding and thickening agents (e.g., gums (such as alginin, guar gum, locust bean gum, and xanthan gum.), pectins, celluloses, (such as methyl cellulose and carboxy methylcellulose), starches, potato flakes, potato flour, flours made from milled or ground grains and legumes (wheat, rice, rye oats barley, buckwheat, corn, lupin, chickpea, lentil, bean etc), antioxidants, surfactants, salts, and nutritional agents e.g. amino acids, such as essential amino acids (e.g. histidine, isoleucine, leucine, glycine, serine, proline, lysine, methionine, phenylalanine, threonine, tryptophan, and valine), di and tri peptides, vitamins (e.g. A, B (1, 2, 3, 5, 6, 7, 9, and 12), C, D, E, K), minerals (calcium, phosphorus, magnesium, sodium, potassium, zinc, iodine, iron, copper.), and phytonutrients, such as carotenoids (e.g. α- and β-carotene, β-cryptoxanthin, lycopene, lutein), flavonoids (e.g. flavanols, flavonols flavones, flavonones, isoflavones,), polyphenols (e.g. anthocyanins, quercetin, ellagic acid)); flavouring agents, such as herbs, spices (e.g. parsley, rosemary, thyme, basil, sage, mint), vegetable flavour (e.g. celery, onion, garlic), yeast extract, malt extract, natural and artificial sweeteners, smoke flavour, amino acids, (e.g. monosodium glutamate), nucleosides, nucleotides and water. One or more ingredients may serve one or more function.

Iron (Fe) may catalyze the chemical reaction of one or more flavour precursor molecules to produce flavouring agents that may impart a desirable flavour and/or aroma such as a meaty, savoury or umami (e.g. beef, chicken, pork, bacon, ham, lamb). Accordingly, the presence of one or more phycobiliproteins, such as phycoerythrin, phycocyanin, allophycocyanin and phycoerythrocyanin, chelated or co-ordinated to Fe (Fe²⁺, or Fe³⁺), in the meat mimetic food product, upon denaturation during the cooking process, may advantageously catalyze reaction of one or more flavour precursor molecules also present in the meat mimetic food product, to produce desirable aromas and flavours.

Some examples of flavour precursor molecules may include (in addition to any of the additional ingredients listed above): sugars, sugar alcohols, sugar acids and derivatives (e.g. glucose, fructose, ribose, sucrose arabinose, inistol, maltose, maltdextrin, galactose, lactose, glucuronic acid, and xylose); oils, such as canola, sunflower, olive, coconut, vegetable, palm, peanut, flaxseed, cottonseed, corn, safflower, rice bran oil; fatty acids, such as caprylic acid, capric acid, lauric acid, mystric acid, palmitic acid, stearic acid, linoleic acid; amino acids, such as cysteine, cystine, leucine, isoleucine, valine, lysine, phenyalanine, threonine, tryptophan, arginine, histidine, alanine, glutamate, asparganine, glycine, proline, serine and tyrosine, and di- and tripeptides, such as glutathione; nucleosides and nucleotides, and vitamins.

In some embodiments, the phycobiliprotein colouring agents may be used in the preparation of a meat mimetic food product such as a ground or shredded meat product, for example, burger patties, kebabs, meat balls, rissoles, meat loaves, sausages, meat sauces and fillings (e.g. chilli, bolognaise, taco fillings, pie fillings), and other formed or shaped meat products (optionally crumbed) such as nuggets, steaks, cutlets, schnitzels, fingers and strips. In some further embodiments the food colouring agent may be used in the preparation of burger patties. In some embodiments the meat mimetic food product is free or substantially free of one or more agents that cause allergic or intolerant reactions, such as MSG, gluten or nuts.

The disclosure will now be further described by reference to the following examples, which are intended for the purpose of illustration only, and are not to be construed as limiting the generality described above.

### EXAMPLES

### Preliminary Evaluation—Effect of Haemoglobin and Myoglobin on Taste and Appearance of Burgers.

Haemoglobin and myoglobin were purchased from Sigma Aldrich. The burger formulation comprised about 20% textured soy protein, about 15% vegetable fat (5% of total formulation weight is coconut fat), about 2.5% fibre, about 5% flavouring (including amino acids) and about 57.5% water.

Using a concentration of 200 mg/100 g burger formulation, haemoglobin and myoglobin were added individually to the burger formulation with flavouring agents, and compared to a burger without added haemoglobin or myoglobin. The raw burger without added haemoglobin or myoglobin was light brown/beige in colour, whereas the other two burgers had a red/brown appearance in the raw state

After cooking, in-house descriptive sensory analysis and flavour analysis by gas chromatography demonstrated that the burgers were essentially identical in nearly all aspects evaluated (e.g. charred appearance, grilled beef odour, smokey charred aftertaste, surface and internal textures, fatty mouthfeel, beef aftertaste, beany/plant taste, saltiness, umami taste, metallic/blood taste and overall beefiness, as well as the presence of sulphur volatiles, aldehydes and pyrazines). The major difference observed between the burgers was with regard to the aspects grilled beef appearance and internal red/bloody appearance, with the burger without added haemoglobin or myoglobin scoring significantly less in these aspects compared to the other two, thereby demonstrating that haemoglobin and myoglobin are responsible for the pink/red (i.e. “bloody”) appearance of the burgers, but do not contribute significantly to flavour profiles/sensory analysis.

### Example 1

Sourcing and Extracting Phycoerythrin from Wild Red Seaweed.

Six red macro algae were collected from the Bellarine Peninsula, Victoria, at 38°16′19.8″S 144°38′27.3″E on Mar. 11, 2019.

Extraction of phycoerythrin comprised the steps of blending the algae in a buffer and centrifugation to remove large particles:

Extraction buffer: 20 mM Sodium Phosphate, pH 7, 0.02% Sodium Azide.
1) Weigh out 10 g red algae into 100 mL extraction buffer.
2) Use Ultra-turrex to homogenise sample and pour through sieve.
3) Clarify at 15,000 RPM in Beckman Coulter Sorvall RC-5 with a F21×50Y fixed angle rotor for 15 mins @ 4 degrees.
4) Concentrate using PES 20 mL 10 kDa cut-off.
5) Dialyse against distilled water to remove metals/contaminants.
6) Freeze-Dry.

The initial homogenised red seaweed produced a red/orange, liquid. Upon clarification by centrifugation the solution became noticeably more fluorescent pink, and this was even more pronounced upon concentration. Freeze-drying produced a darker pink material.

### Thermal Characterisation of R-Phycoerythrin

Thermal characterisation was undertaken to determine if phycoerythrin had a colour change upon heating. First, the crude, homogenised phycoerythrin, before clarification, was heated at 95° C. for 5 min and the colour change visualised. The crude sample turned from red to brown.

After heating at 60° C. and 70° C. for 1 hour, the samples were both stable and showed no colour change. Therefore, to gain a precise measurement of the thermal denaturation temperature of phycoerythrin, differential scanning fluorometry (DSF) was undertaken by heating 100 uL of clarified phycoerythrin from 25-95° C. by increasing the temperature by 0.5° C./10 sec.

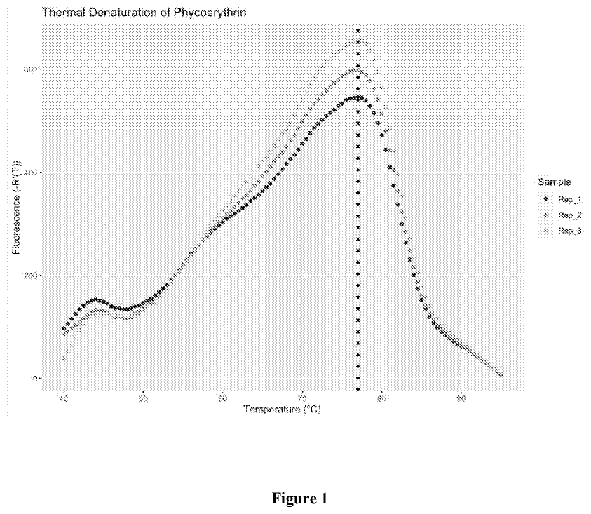

As can be seen in FIG. 1, the thermal denaturation temperature where phycoerythrin loses its fluorescence, and hence has a colour transition, is 77° C.

### Extraction Trial Using Ultrasound

In order to increase the yield of phycoerythrin extraction, ultrasound extraction was investigated. Ultrasound is commonly applied to enhance the recovery of proteins from bacterial and yeast cells. Due to the tough nature of algae cells, ultrasound was applied to enhance phycoerythrin recovery. The following protocol was followed for the extraction:
1) Weigh out 25 g red algae (sample 1) into 150 mL extraction buffer.
2) Homogenize (Ultra-turrex) for 2 mins @ 8000 (min⁻¹).
3) Sonicate @ 160W, 3.3s on 9.9s off, total processing time=5 mins.
4) Clarify at 15,000 RPM in Beckman Coulter Sorvall RC-5 with a F21×50Y fixed angle rotor for 15 mins @ 4° C.
5) Concentrate using PES 20 mL 10 kDa cut-off.
6) Dialyse against 10 L distilled water for 4 hours.
7) Freeze @ −80° C.
8) Freeze dry for 3 days.

The resulting sample was much more blood red in colour following clarification and concentration rather than the fluorescent pink observed for the previous extraction.

To investigate why the sonication extracted solution colour is blood red and not fluorescent pink, absorbance spectrum analysis was undertaken to investigate differences. In addition to characteristic phycoerythrin peaks at approximately 495, 545, 565 nm, the extract obtained using a sonication step showed the appearance of an additional prominent peak at 675 nm and a minor peak at 625 nm that is indicative of allophycocyanin (bluish/green colour) and R-phycocyanin (blue colour), respectively. Mixing a bright pink/red phycobiliprotein (e.g. phycoerythrin) with one or more green/blue phycobiliproteins (e.g. allophycocyanin, phycocyanin) may result in a blood red-coloured extract.

### Example 2

### Manufacture of Phycoerythrin Extracts Suitable for Use as Food Ingredients

To simulate a simple, scalable, food-grade extraction method for obtaining phycoerythrin from red macro seaweed, a modified method using the lab scale method devised in Example 1 was applied:
1—Make up food-grade 200 mM NaCl extraction buffer in tap water.
2—Weigh out 25 g seaweed in 150 mL extraction water.
3—Blend for 2 min in kitchen handheld blender.
4—Sonicate @ 160W, 3.3s on 9.9s off, total processing time=5 mins.
5—Centrifuge to clarify at 5000 g in 4.2r swing rotor.
6—Pour through sieve to remove any large seaweed particles.
7—Freeze at −20° C.
8—Freeze-dry for 3 days and/or until water is completely removed.

Variation in intensity of colour of the liquid extracts was observed, although all were red/pink in colour. Once the seaweed extracts were clarified by centrifugation and filtration, they were then freeze-dried into a powder.

### Use of Phycoerythrin Extracts in Model Ground Meat Product.

Mini burgers were made using the same formulation as that used in the Preliminary Evaluation described supra, with each burger weighing 15 g total. The colouring agent (beetroot, phycoerythrin, haemoglobin or ferritin) and frozen, minced coconut fat (5% w/w) was added to the remaining ingredients and mixed.

The formulations are set out in Table 2-1.

TABLE 2-1

| | Formulation | | | | |
|---|---|---|---|---|---|
| | Dose rate as percent total mass | | | | |
| | Sample number | | | | |
| | 1 | 2 | 3 | 4 | 5 |
| | Sample Name | | | | |
| | | 0.3% | 0.3% | | |
| | Control | Beetroot | PE | Haemoglobin | Ferritin |
| Coconut fat | 5 | 5 | 5 | 5 | 5 |
| Burger | 95 | 94.7 | 94.7 | 93.9 | 94.78 |
| Formulation | | | | | |
| Beetroot | — | 0.3 | — | — | — |
| extract | | | | | |
| Freeze dried | — | — | 0.3 | — | — |
| phycoerythrin | | | | | |
| Vitafit | — | — | — | 1.1 | — |
| Haemoglobin | | | | | |
| CR Ferritin | — | — | — | — | 0.22 |

The burger formulations in Table 2-1 were cooked on a hotplate (Silex Electrogeräte GmbH Germany) at 180° C. for 4 minutes each side, to an internal temperature of 72° C. In a second experiment, burgers were cooked 6 minutes each side, to an internal temperature of 80-85° C. Internal temperature was measured using a digital QM1601 thermometer.

The control burger had a white-yellow appearance when raw, and upon cooking had a brown exterior (due to the Maillard reaction and caramelization), but cooking did not change the internal colour of the burger, which retained the same white-yellow colour of the raw product. The beetroot extract gave both the raw and cooked burger a red appearance, but cooking did not change the internal colour of the burger. The phycoerythrin extract resulted in a “blood”-coloured (pink/red) appearance of the raw product, and a subsequent internal colour change to brown upon cooking. Pooling of red liquid on the burger surface during cooking mimicked the “bleeding” typically observed when animal meat such as beef is cooked. The Vitafit haemoglobin gave the burger a dark brown appearance when raw and a nearly black appearance when cooked. The CR Ferritin-containing burger was identical in appearance to the control burger (raw and cooked).

### Example 3

### Upscale and Characterisation of Phycoerythrin Extraction

To simulate a simple, scalable, food-grade extraction method for obtaining phycoerythrin from wild red macro seaweed (previously collected in Stage 1 of this project), a modified method using the lab scale method devised in Stage 1 was applied:
1—Make up food-grade 20 mM Sodium Phosphate, pH 7.0 extraction buffer in tap water.
2—Weigh out 1000 g seaweed in 5000 mL extraction buffer.
3—Ultra-turrex (homogenise) for 10 mins @ 8000 (min⁻¹).
4—Clarify at 10,000 RPM in Beckman Coulter Sorvall RC-5 with a F21×500Y fixed angle rotor for 15 mins @ 4T.
5—Pour through sieve to remove any large seaweed particles.
6—Concentrated 3.3× using a SM-PES 20,000 Da MWCO Synder ultrafiltration membrane before diafiltration with MilliQ water 7× to remove residual seaweed aromas.
7—Freeze at −20° C.
8—Freeze-dry for 3 days and/or until water is completely removed.

The wild red seaweed for this process was collected at Dromana Beach, Victoria, Australia on 31 Dec. 2019. Analysing the absorbance spectrum of the pre- and post-ultrafiltration samples shows the characteristic peaks for phycoerythrin and shows that the filtration process concentrates the phycoerythrin relative to the protein peak at 280 nm. Running these samples on an SDS-PAGE gel also shows only one protein band, showing the sample is pure for the phycoerythrin protein

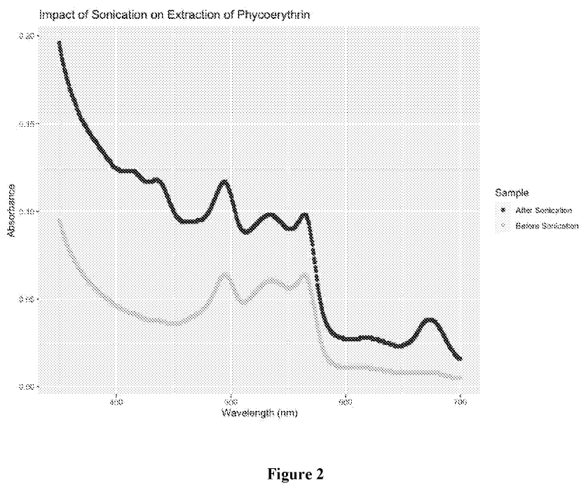

The upscaled phycoerythrin extract was heated at 95° C. for 6 min, with a colour change from bright pink to brown observed. The absorbance spectrum for the two samples show that upon heating the characteristic peaks for phycoerythrin are highly diminished and that the peaks broaden, indicating that the colour change is due to a change in the protein structure of the phycoerythrin (see FIG. 2).

### Example 4

### Characterization and Comparison of Various Phycoerythrin Extracts

R-Phycoerythrin was extracted from the following algal species, using the methods described above in Example 1.
(a) Porphyridium purpureum (Strain CS-25, University of Technology, Sydney)
(b)Asparagopsis taxiformis (CH4Global)
(c) Bonnemaisonia hamifera (CH4Global)
(d) Wild Seaweed sample collected from the beach.

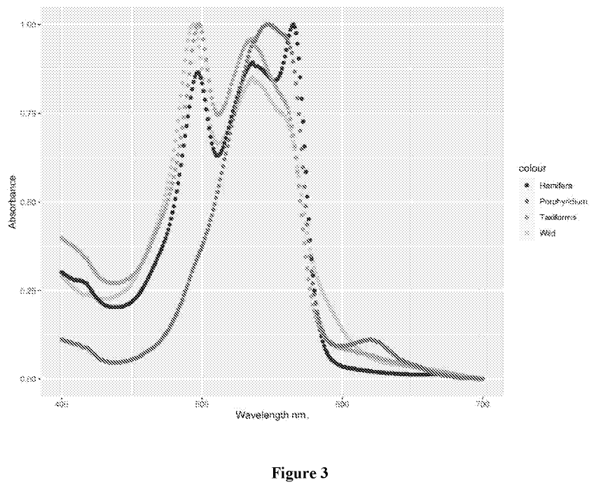

UV Absorbance spectra were recorded for each Phycoerythrin sample. The results are depicted in FIG. 3. It is noted that a peak at about 495-500 nm is observed for each of samples (b)-(d), consistent with the phycouribilin chromophore bound to phycoerythrin, but that this peak is essentially absent for sample (a). The differences observed reflect the subtypes found in nature, which is dependent on the number, arrangement and types of protein subunits that make up the phycoerythrin protein.

### Thermal Denaturation

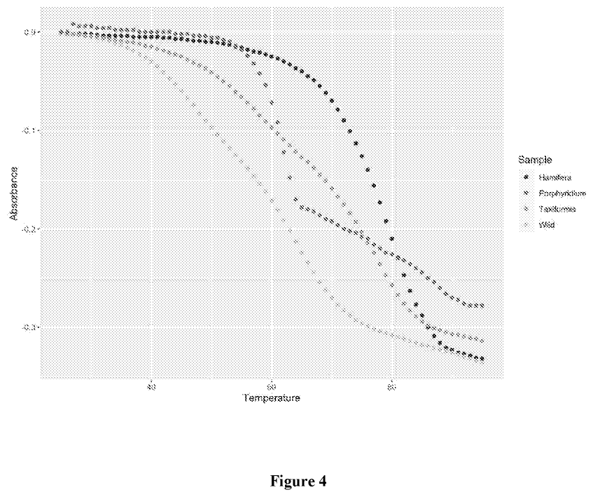

Thermal denaturation of each Phycoerythrin sample (a)-(d) was undertaken. The results are depicted in FIG. 4.

Visually, all the phycoerythrin samples demonstrated loss of colour upon heating to 95° C. However, the phycoerythrin extracted from Porphyridium purpureum retained more of its colour compared to the others. This is most likely due to the particular subtype of phycoerythrin found in Porphyridium purpureum.

### Example 5

Manufacture of Phycoerythrin Extracts Suitable for Use as Food Ingredients from Micro Algae Grown in Culture

To simulate a simple, scalable, food-grade extraction method for obtaining phycoerythrin from biomass of red micro algae grown in culture (CS-174 Rhodomonas salina, (University of Technology Sydney)), a modified method based on using the lab scale method devised in Example 2 was applied.
1. Thaw frozen biomass (dry weight* content of algae=50 mg/g of wet biomass) ≠*Reference to a dry weight basis refers to algae with all water removed
2. Add water to culture biomass at a ratio of 2.75 mL of water per g (wet weight) of biomass.
3. Blend for 1 min, 10,000 rpm (Ultra-turrax, model T8, IKA/Janke & Kunel GmbH Germany).
4. Clarify by centrifugation: 5 min, 4000 g (Beckman J6-MI centrifuge, JS 4.2 rotor.).
5. Decant clear supernatant as crude liquid extract.
6. Clarify crude extract by centrifugation: 15 mins 10,000 RPM, 4° C. (Sorvall RC-5 centrifuge F21×500Y rotor).
7. Decant clear supernatant as aqueous food grade Phycoerythrin Extract.

### Example 6

Characterisation of Phycoerythrin Extracts from Micro Algae Grown in Culture by UV/Spectroscopy

To identify if a particular extract is appropriate to be used as a heat-sensitive food colourant and the relative purity of the extract, the UV/Visible spectrum of the extract may be obtained using typical laboratory equipment. The heat sensitivity of the compounds of interest may be obtained by measuring the response of the extract to heat at key wavelengths. To confirm the modification of the colour profile, a further UV/Visible spectrum may be obtained after heating the extract.

Identification of UV/Visible spectrum of extracts.
1. A test solution was prepared by diluting the liquid extract with water to obtain a reading within the working range of the instrument. In this case a 1/10 dilution was sufficient.
2. Prepare the UV spectrometer (UV-1700 Shimadzu Australia) to measure a wavelength scan with the following measurement properties.
   a. Wavelength Range (nm.): 270.00 to 700.00
   b. Scan Speed: Medium
   c. Sampling Interval: 1.0s
   d. Auto Sampling Interval: Disabled
   e. Scan Mode: Single Wavelength range
3. Run the scan and collect the data
4. Prepare the UV spectrometer (UV-1700 Shimadzu Australia) to measure a wavelength scan at wavelengths of interest identified at step 3, with the following measurement properties.
   a. Start Temperature 20° C.
   b. Start wait 10 sec
   c. Ramp rate 2.0° C./min
   d. Measure wait 5 sec.
   e. Interval 1° C.
   f. End temp (C) 95
5. Run the scan and collect the data
6. Re-run the wavelength scan with the settings used at step 2
7. Run the scan and collect the data

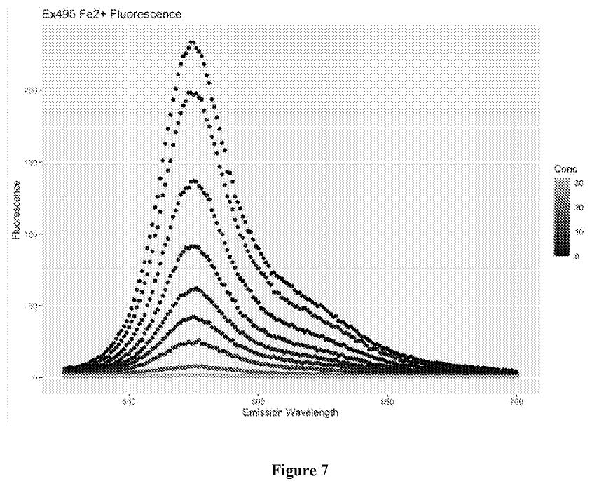

The data obtained for the extract described in Example 6 are shown in FIG. 7 (UV/VIS absorbance spectrum) and FIG. 8 (temperature scan). The extract shows a major peak at approximately 550 mn which is characteristic of phycoerythrin. The ratio of the absorbance at the λₘₐₓ peak, compared to the absorbance at 280 nm (corresponding to absorbance of protein) is 2.7:1, indicating a high proportion of extracted protein as phycoerythrin.

A temperature scan at 550 nm indicated a 50% loss of absorbance at approximately 63° C., with a total colour loss was about 20% of initial. A small residual peak was present when the heated extract was re-run in a wavelength scan.

### Example 7

Application of “Food Grade” Phycoerythrin Extracts from Micro Algae Grown in Culture in Meat Mimetic Food Products: Burger Patties Simulating White Meat Products Such as Chicken and Red Meat Products Such as Beef.

The aqueous phycoerythrin extract from Example 6 was used to formulate burger patties that simulate the properties of red and white meat products. Using the formulations shown below in Table 8-1:

TABLE 7-1

| | Percentage by wet weight | |
|---|---|---|
| | White meat | Red meat mimetic |
| Ingredient | mimetic burger | burger |
| Aqueous extract from Example 5 | 3 | 10 |
| Colour (Caramelised Sugar) | Nil | 0.3 |
| Thickeners (Modified Cornstarch, Soy Isolate, | 3.3 | 3.3 |
| Methylcellulose) | | |
| Textured Soy Protein Concentrate | 23 | 23 |
| Flavours (contains Glutamic Acid) | 3.1 | 3.1 |
| Canola Oil | 10 | 10 |
| Coconut Fat | 5 | 5 |
| Water | 52.6 | 45.3 |

The white meat mimetic demonstrated an appropriate colour for white meats as raw product. A colour change characteristic of the transition from raw to cooked product was observed in the temperature range of 68 to 70° C. No adverse flavour impacts were noted on sensory evaluation and the formulation adjudged suitable for use

For the red meat mimetic burger, a suitable raw beef colour was achieved through additional incorporation of burnt sugar and adjusting the level of aqueous phycoerythrin extract proportion. A colour change characteristic of the transition from raw to cooked product was observed in the temperature range of 68 to 70° C. No adverse flavour impacts were noted on sensory evaluation and the formulation adjudged suitable for use

### Example 8

Application of Whole Biomass from Micro Algae Grown in Culture in Meat Mimetic Food Products: Burger Patties Simulating White Meat Products, Such as Chicken, and Red Meat Products, Such as Beef.

Whole (defrosted frozen) micro-algae (CS-174 Rhodomonas salina, (University of Technology Sydney)) was used to formulate burger patties that simulate the properties of red meat products, using the formulations shown in Table 8-1 below:

TABLE 8-1

| | Percentage by wet weight | |
|---|---|---|
| | White meat | Red meat mimetic |
| Ingredient | mimetic burger | burger |
| “wet” biomass of Rhodomonas salina | 2% | 5% |
| (dry weight of algae = 50 mg/g of wet biomass) | (0.1%) | (0.25%) |
| Colour (Caramelised Sugar) | Nil | 0.3 |
| Thickeners (Modified Cornstarch, Soy Isolate, | 3.3 | 3.3 |
| Methylcellulose) | | |
| Textured Soy Protein Concentrate | 23 | 23 |
| Flavours (contains Glutamic Acid) | 3.1 | 3.1 |
| Canola Oil | 10 | 10 |
| Coconut Fat | 5 | 5 |
| Water | 53.6 | 50.3 |

The red meat mimetic demonstrated an appropriate colour as raw product.

Burger patties were cooked on a commercial hotplate. Internal temperature was monitored using a probe thermometer and colour change was observed visually.

A colour change characteristic of the transition from raw to cooked product was observed in the temperature range of 68 to 70° C. Sensory evaluation indicated a slightly enhanced umami flavour notes and no adverse marine flavour taints.

### Example 9

### Iron Binding by Purified Phycoerythrin Red Seaweed Extract

Phycoerythrin extract from Example 1 (extraction included ultrasound treatment) was dissolved at a concentration of 2 mg/mL in 20 mM sodium phosphate buffer, pH 7 containing 0.02% sodium azide. Iron (II) chloride was dissolved in the same buffer at an initial concentration of 100 mM. The dissolved phycoerythrin extract was mixed 1:1 with a concentration series of iron chloride to give a final protein concentration of 1 mg/mL and final iron chloride concentrations of 0, 0.25, 0.5, 1, 2, 4, 8, 16 and 32 mM.

A fluorescence emission scan from 515-700 nm using an excitation wavelength of 498 nm was then performed on all samples using a Thermofisher Varioskan Flash (Instrument version 4.00.52). R-phycoerythrin has an emission maximum of 575 nm and therefore if iron binding occurs with the linear tetrapyrrole moiety of the protein, a change in fluorescence will be observed. FIG. 7 shows the fluorescent iron binding results. As can be seen, there is a decrease in phycoerythrin fluorescence with increasing iron concentration, indicative that iron is binding to the linear tetrapyrrole of the protein and that phycoerythrin can coordinate with iron and hence be an iron carrier protein.

### Example 10

### Evaluation of Bioavailability of Iron Bound to Phycoerythrin.

Bioavailability of phycoerythrin-bound iron was evaluated using an established human intestinal model—Caco-2/HT29-MTX-E12 transwell model. Intestinal uptake of iron with and without phycoerythrin was measured through human ferritin formation.

### Test Solutions

Iron (II) chloride (ferrous chloride, FeCl₂),
Iron (III) chloride (ferric chloride, FeCl₃) and
Iron (II) sulphate (ferrous sulphate, FeSO₄·7H₂O)
Phycoerythrin

### Methods

Human Caco-2(enterocytes) and HT29-MTX-E12 (goblet) cells grown on a semi-permeable membrane comprise the intestinal barrier model. A co-culture was grown instead of a single cell line as the in vivo intestinal barrier contains several different cell types. To ensure that any treatment effect in the intestinal barrier model is not related to cytotoxicity, Caco-2/HT29-MTX-E12 cell viability was measured in response to the kiwifruit digesta.

Caco-2/HT29-MTX-E12 cell viability was measured in response to all samples to determine treatment concentrations for the intestinal barrier model. The use of non-cytotoxic sample concentrations ensures that any treatment effect in the intestinal cell assay is not related to cytotoxicity. Cell viability was measured using the CyQUANT Cell Proliferation Assay to estimate cell viability as described below:
9×10⁴ Caco-2 cells and 1×10⁴ HT29-MTX-E12 cells were plated into 96 well black plates and incubated for 7 days at 37° C. with 5% CO₂.
After 7 days, growth media (DMEM, 10% fetal bovine serum) was removed, and Hank's balanced salt solution (HBSS) buffer was used to wash the cells (via robot).
Project samples were prepared in HBSS and added to cells using a multichannel pipette. Cells were incubated overnight at 37° C. with 5% CO₂.
Treatments were removed after 16-18 hours and washed with HBSS before CyQUANT reagent diluted in HBSS buffer was applied to the cells (using a robot).
After one hour, fluorescence was measured with excitation 485 nm and emission 530 nm.

Human Caco-2(enterocytes) and HT29-MTX-E12 (goblet) cells grown on a semi-permeable membrane comprise the intestinal barrier model. The co-cultures were grown on transwells as described below:
Caco-2 cells and HT29-MTX-E12 cells flasks were passaged when ˜90% confluent.
Cells were counted using a haemocytometer (or Coulter counter) to determine cell number/mL.
0.6 mL growth media (no cells) was added to basolateral chamber of transwells.
0.2 mL Caco-2/HT29-MTX-E12 cell solution was carefully added into apical chamber to achieve 3.6×10₄ Caco-2 and 4×10³ HT29-MTX cells.
Co-cultures were grown on transwells for 21 days with media changed every 2-3 days.
At 21 days transepithelial electric resistance (or TEER) was measured using a Millicell voltohmeter from the apical to basolateral chamber (FIG. 1). These measurements indicate integrity of cell layer ensuring the cells are polarised and an intact barrier is ready for experimentation. All TEER measurements were above 280 Ω·cm² and indicative of differentiated cells and an intact barrier. Following preparation of an intact intestinal cell barrier, impact of ferrous chloride, ferric chloride, and ferrous sulphate was observed on intestinal barrier function as described:
All transepithelial electrical resistance (TEER) readings were measured at day 21 and recorded prior to application of sample treatments.
Iron samples and phycoerythrin were prepared in HBSS at non-cytotoxic concentrations.
Growth media was removed from the cells and replaced with HBSS for 2 hours to deplete cells of fetal bovine serum (present within growth media).
HBSS was removed and replaced with sample treatments for 2 hours. Treatments were removed, replaced with HBSS, and incubated overnight.
After 16-18 hours cells (on apical side) were washed with PBS before trypsin was applied to remove cells from transwell membranes.
Cells were collected by centrifuging for 5 minutes.
Abcam ferritin assay was conducted according to manufacturer's instructions.

Results from the ferritin assay were analysed for significant differences using unpaired t tests. Differences were deemed significant when P<0.05. All statistical analyses were performed using GraphPad Prism 5 software.

### Results

Cell viability was measured in response to iron solutions with and without 8 mg/mL phycoerythrin. 100, 50, and 25 mm iron solutions with 8 mg/mL phycoerythrin exhibited greater than 80% cell viability and were tested in the intestinal model.

All samples treatments were performed in the presence of 80 μM ascorbic acid. Previous studies involving ferritin formation in intestinal cell models use ascorbic acid to improve intestinal uptake of iron (Mahler et al. Characterization of Caco-2 and HT29-MTX cocultures in an in vitro digestion/cell culture model used to predict iron bioavailability, Journal of Nutritional Biochemistry; 20:494-502, 2009.) and mimic biological levels of ascorbate (50 and 100 μM) normally present in humans (Badu-Boateng, C. and Naftalin, R. J. Ascorbate and ferritin interactions: Consequences for iron release in vitro and in vivo and implications for inflammation, Free Radic Biol Med., 133:75-87, 2019).

Although no significant differences were observed between cells treated with 100 μM iron solutions and ascorbic acid compared to cells treated with iron solutions, ascorbic acid, and phycoerythrin, in the absence of iron, phycoerythrin plus ascorbic acid produced similar ferritin production compared to all other iron solutions. It is possible that the initial treatment concentrations of iron (100 μM) and phycoerythrin (8 mg/mL) were too high to observe synergism between the two components.

Cells were treated with 25 μM or 50 μM iron solutions, and 4 or 8 mg/mL phycoerythrin. The results are presented in Table 10-1.

| Different Fe | Without | With 4 mg/mL | With 8 mg/mL |
|---|---|---|---|
| solutions | phycoerythrin | phycoerythrin | phycoerythrin |
| 50 μM Fe solutions | Ferritin (ng/mL) | | |
| Fe (II) chloride | 5.2 ± 1.6 | 10.1 ± 3.5 | 16.0 ± 5.9 |
| Fe (III) chloride | 1.7 ± 0.8 | 13.0 ± 3.1* | 1.8 ± 0.6 |
| Fe (II) sulphate | 3.9 ± 0.8 | 9.8± 2.0* | 11.7 ± 1.7* |
| No Fe | — | — | 2.5 ± 0.4 |
| 25 μM Fe solutions | Ferritin (ng/mL) | | |
| Fe (II) chloride | 4.4 ± 1.0 | 15.8 ± 1.7* | 13.9 ± 1.8* |
| Fe (III) chloride | 3.3 ± 0.5 | 12.7 ± 0.9* | 12.7 ± 1.1* |
| Fe (II) sulphate | 4.1 ± 0.5 | 17.8 ± 1.4* | 11.1 ± 1.3* |
| No Fe | — | 12.2 ± 0.3# | 7.5 ± 1.6\hat{ } |

Note:
*denotes a significant difference between cells treated with iron solutions compared to cells treated with iron and 4 mg/mL or 8 mg/mL phycoerythrin solutions.
#denotes a significant difference between cells treated with iron solutions compared to cells treated with 4 mg/mL phycoerythrin solutions.
\hat{ }denotes a significant difference between cells treated with Fe (III) chloride solutions compared to cells treated with 8 mg/mL phycoerythrin solutions.
Statistical differences were determined using unpaired t tests (GraphPad Prism 5).

Co-treatment of cells with 4 mg/mL phycoerythrin and 50 μM ferric chloride or ferrous sulphate, significantly improved ferritin production compared to cells only treated with ferric chloride or ferrous sulphate. Co-treatment with 8 mg/mL phycoerythrin significantly increased ferritin production in the presence of ferric sulphate.

Co-treatment of cells with 4 or 8 mg/mL phycoerythrin and 25 μM ferrous chloride, ferric chloride, or ferrous sulphate, significantly improved ferritin production compared to cells only treated with the different iron solutions. Treatment with only 4 mg/mL phycoerythrin (i.e. no added iron) also significantly increased ferritin production compared to treatment with iron solutions. Similarly, treatment with only 8 mg/mL phycoerythrin (i.e. no added iron) significantly increased ferritin but only compared to treatment with ferric chloride.

Phycoerythrin can improve bioavailability of iron and promote production of ferritin in vitro. Inclusion of phycoerythrin in food products may improve intestinal uptake of iron and ferritin production, particularly when combined with lower levels of iron.

### Example 11

### An Exemplary Method for Quantification of R-PE Content of Biomass

Method adapted from the book chapter: “Extraction and Purification of R-phycoerythrin from Marine Red Algae” by Justine Dumay, Michèle Morançais, Huu Phuo Trang Nguyen, and Joël Fleurence in “Natural Products From Marine Algae: Methods and Protocols, Methods in Molecular Biology, vol. 1308”, by Dagmar B. Stengel and Solene Connan (eds.), Springer Science Business Media New York 2015, DOI 10.1007/978-1-4939-2684-8_5,

The below method exemplifies calculations based on absorption peaks at 495 and 565 nm, however, it will be understood that corresponding calculations can be performed for PE samples demonstrating corresponding peaks in the ranges of 495-503 nm (e.g., 495, 496, 497, 498, 499, 500, 501, 502 or 503 nm) and 540-570 nm (e.g, about 540, 545, 550 555, 560, 565, 570 nm).
1. Weigh accurately approximately 1 g biomass into a 10 mL graduated centrifuge tube
2. Add deionised water to approximately 5 mL
3. Homogenise for 30 seconds with a high shear mixer (UltraTurrax T8 speed 6) while keeping the tube cold (ice bath)
4. Make up to the 10 mL mark with deionised water
5. Mix for 30 minutes at 4 degrees
6. Centrifuge 20 minutes 4000 g, 4 degrees
7. Decant supernatant into a 25 mL volumetric flask
8. Add deionised water to the pellet to approximately 5 mL
9. Homogenise for 30 seconds with a high shear mixer (UltraTurrax T8 speed 6) while keeping the tube cold (ice bath)
10. Make up to the 10 mL mark with deionised water
11. Mix for 30 minutes at 4 degrees
12. Centrifuge 20 minutes 4000 g, 4 degrees
13. Combine the supernatant with the first extract in the 25 mL volumetric flask
14. Make up to the 25 mL mark with deionised water
15. Measure the absorbance values between 350 and 750 nm.

The Phycoerythrin content (mg/mL) content can be estimated following the Beer and Eshel equation (Beer S., and Eshel A., (1985) Determining phycoerythrin and phycocyanin concentrations in aqueous crude extracts of red algae. Aust J Mar Freshw Res 36:785-793):

$$PE = [ ( {A}_{565} - {A}_{592} ) - ( {A}_{495} - {A}_{592} ) \times 0.2 ) ] \times 0.12 .$$

1.-24. (canceled)

25. A meat mimetic food product comprising one or more phycobiliproteins in an amount sufficient to visually confer a pink or red colour to the food product, and which affords a visual colour change upon cooking of the food product to an internal temperature in the range of about 50-95° C., wherein the one or more phycobiliproteins are obtained from microalgae.

26. The meat mimetic food product according to claim 25 wherein the visual colour change occurs upon cooking of the food product to an internal temperature in the range of about 60-85° C.

27. The meat mimetic food product according to claim 25 wherein the one or more phycobiliproteins comprise R-phycoerythrin, and/or C-phycoerythrin, and/or B-phycoerythrin and/or b-phycoerythrin.

28. The meat mimetic food product according to claim 25 wherein the one or more phycobiliproteins comprise phycoerythrin in an amount of at least 50%, or at least 70% or at least 90%, or at least 99% of all phycobiliproteins on a w/w basis.

29. The meat mimetic food product according to claim 25 wherein the one or more phycobiliproteins are present in the form of an extract, or purified or at least partially purified isolate from microalgae.

30. The meat mimetic food product according to claim 25 wherein the one or more phycobiliproteins are present in the form of whole or macerated microalgae.

31. The meat mimetic food product according to claim 25 wherein the microalgae is a species selected from Rhodophyceae, and/or Cyanophyceae and/or Cryptophyceae,

32. The meat mimetic food product according to claim 31 wherein the microalgae is a species selected from Rhodophyceae.

33. The meat mimetic food product according to claim 31 wherein the microalgae is a species selected from Cyanophyceae.

34. The meat mimetic food product according to claim 31 wherein the microalgae is a species selected from Cryptophyceae.

35. The meat mimetic food product according to claim 25 wherein the microalgae is selected from Porphoridium sp, Rhodella sp., Rhodosorus sp., Arthrospira sp, Fremyella sp. or Rhodomonas sp,

36. The meat mimetic food product according to claim 35 wherein the microalgae is selected from a Porphyridium sp or a Rhodomonas sp.

37. The meat mimetic food product according to claim 35 wherein the microalgae is Rhodomonas salina.

38. The meat mimetic food product according to claim 25 wherein the phycobiliprotein demonstrates a 50% absorbance reduction of λₘₐₓ in the range of about 50-95° C.

39. The meat mimetic food product according to claim 38 wherein the phycobiliprotein demonstrates a 50% absorbance reduction of λₘₐₓ in the range of about 60-85° C.

40. The meat mimetic food product according to claim 38 wherein λₘₐₓ is in the range of about 540-570 nm.

41. The meat mimetic food product according to claim 25 wherein the phycobiliprotein is phycoerythrin and exhibits a 540-570 nm to 495-503 nm UV/visible absorbance peak ratio of at least 1:1, or at least 3:1, or at least 5:1, or at least 7:1, or at least 10:1, preferably only a single peak at 540-570 nm, more preferably at 550-565 nm.

42. The meat mimetic food product according to claim 30 wherein, the microalgae is included in an amount of 0.1 to 20% w/w on a dry weight basis, preferably 0.1 to 10% w/w, more preferably about 0.1 to 5% w/w.

43. The meat mimetic food product according to claim 25, wherein the microalgae has a phycoerythrin content from about 1-150 mg/g dry weight, preferably about 40-80 mg/g dry weight.

44. The meat mimetic food product according to claim 25, further comprising one or more additional colouring agents having a colour selected from red, magenta, purple/violet, orange, yellow, brown blue and green.

45. The meat mimetic food product according to claim 25, further comprising one or more additional colouring agents selected from betalains, carotenoids, flavonoids, polyphenols, caramel, burnt sugar, and a juice, concentrate, extract or dried powder from derived from plants, including berries, grapes, beetroot, radish, turmeric and carrot.

46. The meat mimetic food product according to claim 25 comprising a non-animal protein source, one or more carbohydrates, one or more fats and oils, one or more flavour components and water.

47. The meat mimetic food product according to claim 46, wherein the non-animal protein source is a plant based protein selected from soy, faba bean, pea, wheat, chickpea and mungbean protein.

48. The meat mimetic food product according to claim 25 which is a chicken, beef, lamb, veal, pork, goat, kangaroo or fish/seafood mimetic product.

49. The meat mimetic food product according to claim 25, which is a ground or shredded meat product, or a formed or shaped meat product.

50. The meat mimetic food product according to claim 25, which has been cooked to an internal temperature in the range of about 50-95° C., preferably 60-85° C.
